(19) United States
(12) Patent Application Publication
(10) Pub. No.: US 2004/0171005 A1
(43) Pub. Date: Sep. 2, 2004

(54) Codon-optimised nucleic acid coding for apoaequorin and uses thereof

(76) Inventors: John Andrew Printen, Macclesfield (GB); Wendy Irene Vernon, Macclesfield (GB)

Correspondence Address: FISH & RICHARDSON PC, 225 FRANKLIN ST, BOSTON, MA, 02110, US

(21) Appl. No.: 10/475,640

(22) PCT Filed: Apr. 24, 2002
(86) PCT No.: PCT/GB02/01900

(30) Foreign Application Priority Data
Apr. 27, 2001 (SE) 0101519-7

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention relates to a codon-optimised nucleic acid sequence coding for apoaequorin polypeptide and uses thereof.

The present invention is directed to improved luminescent assay systems, to methods for increasing expression, sensitivity and magnitude of the jellyfish photoprotein aequorin.

The luminescent jellyfish Aequorea victoria contains a photoprotein, aequorin, which has been used extensively as a biological calcium indicator in cells (Satoshi Inouye et al., Proc.Natl. Acad Sci (USA) 82:3154-3158, 1995). Detection of calcium flux can lead to information regarding important modulators and physiological mechanisms within a cellular environment.

The aequorin complex consisting of a 22,000MW apoaequorin protein, molecular oxygen and the lipophilic prosthetic luminophore coelenterazine, emits blue light (λmax=470 nm) when bound to calcium ions. When three calcium ions bind to this complex coelenterazine, the functional chromophore in aequorin, is oxidised to coelenteramide with a concomitant release of carbon dioxide and blue light. This allows the measurement of Ca²+ concentrations from 0.1 um to >100 um.

Aequorin has been used for many years as a reporter of changes in intracellular calcium concentration in host cells, such as mammalian or Xenopus oocytes. Initially this was undertaken by microinjection of purified aequorin protein into the host/assay cell. More recently, the cloning of the aequorin gene has enabled recombinant expression of this protein within the host/assay cell. Such systems are useful in screening for small molecules effecting the activation state of receptors and/or ion channels.

While aequorin has been extensively used to assay changes in intracellular calcium concentration in mammalian cells, use has been limited to cells in which plasmids containing the apoaequorin gene can be easily introduced. This is usually accomplished by standard transfection techniques, such as complexing the DNA with ionic lipid reagents or precipitation with CaPO₄. Restricted use in easily transfected cell types is due to low protein expression of the native Aequoria victoria apoaequorin cDNA and weak light emission in calcium assays when only a small population of cells are harbouring an apoaequorin expression plasmid. Clearly, increased intracellular expression of apoaequorin would lead to a more robust luminescent output and greater signal-to-noise ratio in calcium assays. In addition, increased translation of apoaequorin cDNA would allow calcium assays to be performed in mammalian cells refractory to standard transfection techniques. This could be accomplished by introducing apoaequorin cDNA by retroviral gene delivery, or other methods which usually result in a lower cDNA copy number than transient transfections, but allow gene delivery to a wider range of mammalian cell types.

Certain Prior Art:

The amino acid sequence of the photoprotein aequorin was first published by Charbonneau et al. (Biochemistry 24:6762-6771, 1985) and is disclosed in the EMBL sequence database under Accession number: M11394 (also disclosed as SEQ ID No. 1 herein).

U.S. Pat. No. 5,874,304 and U.S. Pat. No. 6,020,192 (Univ. Florida Res. Found. Inc.) relates to the enhanced expression of the photoprotein, green fluorescent protein (GFP), by expression in a recombinant mammalian system of codon-optimisation (humanisation) gene(s).

U.S. Pat. No. 5,422,266; U.S. Pat. No. 5,766,941; U.S. Pat. No. 5,798,441; U.S. Pat. No. 5,744,579 and U.S. Pat. No. 5,162,227 (Univ. Georgia Res. Foundation) relates to the cloning, sequencing and recombinant expression of apoaequorin in microorganisms, and lay claim to wild-type and various mutant forms of apoaequorin and nucleic acids coding therefore. Although they point to the well established degeneracy of the genetic code and claim all possible degenerate codon combinations, no codon-optimised versions of the variant genes is made, proposed or disclosed. Nor is there any teaching of the potential benefit, in terms of enhanced expression and magnitude of luminescent output, that can be obtained with a codon-optimised gene, as described in the present invention.

EP-A-0341477 (Chisso Corp.), relates to a process for producing aequorin in a mammalian cell system. Again, only the published wild-type apoaequorin cDNA sequence is used and there is no teaching in this specification of the use of a codon-optimised version of the apoaequorin gene, nor of the benefit in terms of enhanced expression and magnitude of luminescence that can be obtained with a codon-optimised gene as described in the present invention.

EP-A-0264819 (Chisso corp.), disclose various variant forms of the natural aequorin gene (pAQ440) and teach their expression in Escherichia coli. Using such genes they have demonstrated the ability to produce apoaequorin from which regeneration of aequorin is possible without needing the presence of 2-mercaptoethanol. There is no mention of codon-optimisation in this specification.

U.S. Pat. No. 5,360,728 and U.S. Pat. No. 5,541,309 (W.H.O.I), disclose modified apoaequorin having increased bioluminescent activity. Although they point to the well established degeneracy of the genetic code and claim all possible degenerate codon combinations, no codon-optimised versions of the variant genes is made, proposed or disclosed. Nor is there any teaching of the potential benefit, in terms of enhanced expression and magnitude of luminescence output, that can be obtained with a codon-optimised gene, as described in the present invention.

U.S. Pat. No. 5,714,666 (Children's Hosp. of Philadelphia; Trustees of Univ. Pennsylvania), is directed to a transgenic mouse whose neuronal cells comprise a gene encoding apoaequorin. In the examples, only the published wild-type apoaequorin cDNA sequence is used. There is no teaching in this specification of the use of a codon-optimised version of the apoaequorin gene.

It has been reported that enhanced levels of recombinant protein expression may be obtained by adapting the gene of interest to possess codons that are favoured by the particular host being used. What is not at all established, is that the magnitude of the luminescent signal generated by recombinantly expressed aequorin can be greatly enhanced when a codon-optimised sequence is used for expressing apoaequorin in a mammalian cell system. The Examples herein, show that when transiently transfected cells are stimulated with the calcium ionophore, Ionomycin, the magnitude of luminescent output is much greater (@>20-fold; see Table 1) than the increased aequorin expression level (@ 8-fold), as measured by western blotting against the HA epitope fused to the amino terminus.

The inventors are not aware of any publication which teaches use of a codon-optimised version of apoaequorin gene for increased expression of the apoaequorin protein in a mammalian cell, with the added benefit of greatly enhanced magnitude of luminescent signal.

The present invention arises from the discovery that humanisation of the aequorin gene provides a vast improvement over the traditional aequorin constructs in detecting intracellular calcium flux.

According to a first aspect of the invention there is provided a codon-optimised nucleic acid sequence coding for apoaequorin polypeptide.

In a preferred embodiment the codon-optimised apoaequorin polypeptide has the sequence depicted in SEQ ID No. 1, or a truncated version thereof.

A truncated version is one wherein one or more amino acids at or close to the N- or C-terminus of the protein are absent. In one embodiment the truncated version has fewer than 50 amino acids removed from the C-terminus. The truncated version must retain some luminescent property.

A further embodiment encompassed variant forms of the apoaequorin protein, such as for example, those described in U.S. Pat. No. 5,360,728 or EP-A-0264819, which variant forms may possess enhanced or altered luminescent properties and whose sequence is generally based on that depicted in SEQ ID No. 1. Preferably the variant form possesses only one or a few amino acid changes from that of the wild-type sequence. Three examples of variant forms of apoaequorin are those in which the aspartic acid amino acid at position 124 is substituted for by serine, the glutamic acid amino acid at position 135 is substituted for by serine and the glycine at amino acid 129 is substituted for by alanine. A variant sequence will possess, in increasing order of preference, at least 80%, 85%, 90%, 95%, 97%, 98% and 99% sequence identity with the sequence depicted in SEQ ID No. 1. Sequence identity between two sequences can be assessed using best-fit computer alignment analysis using suitable software such as Blast, Blast2, NCBI Blast2, WashU Blast2, FastA, Fasta3 and PILEUP, using a scoring matrix such as Blosum 62. Such software packages endeavour to closely approximate the “gold-standard” alignment algorithm of Smith-Waterman. Thus, the preferred software/search engine programme for use in assessing the percent identity, i.e how two primary polypeptide sequences line up is Smith-Waterman.

As used herein, the term “codon-optimised” or “humanised” means a nucleic acid protein coding sequence which has been adapted for expression in mammalian, particularly human, cells by substitution of one or more, preferably a significant number of jellyfish apoaequorin codons with codons that are more frequently used in human genes.

In another preferred embodiment, the percentage of humanised codons is, in increasing order of preference, at least 10%, 20%, 30%, 40%, 50%, 60%, 70%, 80%, 90%, 95%, 98%, 99%. In a particular embodiment each and every apoaequorin codon position is humanised.

The codon-optimised sequences of the present invention are generally cDNAs, although genomic copies are also encompassed.

The codon-optimised sequences can be synthesised chemically using standard techniques in the art. Alternatively, wild-type genomic or cDNA can be mutated. Nucleotide changes or mutations may be introduced into a polynucleotide sequence by de novo polynucleotide synthesis, by PCR, by site directed mutagenesis using appropriately designed oligonucleotide primers or by any other convenient means know to the person skilled in the art. The termini of the humanised apoaequorin gene may be engineered to possess suitable restriction enzyme recognition sequences, or may be flanked by additional nucleic acid portions that comprise suitable restriction enzyme recognition sequences, so as to facilitate cloning of the humanised gene into plasmid vectors, and the like.

A variety of mammalian expression vector/host systems may be used to express the codon-optimised apoaequorin coding sequence. Particular examples include those adapted for expression using a recombinant adenoviral, adeno-associated viral (AAV) or retroviral system. Vaccinia virus, cytomegalovirus, herpes simplex virus, and defective hepatitis B virus systems, amongst others may also be used. Although it is preferred that mammalian expression systems are used for expression of the humanised apoaequorin gene, it will be understood that other vector and host cell systems such as, bacterial, yeast, plant, fungal, insect are also possible.

Expression vectors usually include an origin of replication, a promoter, a translation initiation site, optionally a signal peptide, a polyadenylation site, and a transcription termination site. These vectors also usually contain one or more antibiotic resistance marker gene(s) for selection. Suitable expression vectors may be plasmids, cosmids or viruses such as phage or retroviruses. The coding sequence of the polypeptide is placed under the control of an appropriate promoter (i.e. HSV, CMV, TK, RSV, SV40 etc), control elements and transcription terminator so that the nucleic acid sequence encoding the polypeptide is transcribed into RNA in the host cell transformed or transfected by the expression vector construct. The coding sequence may or may not contain a signal peptide or leader sequence for secretion of the polypeptide out of the host cell. Preferred vectors will usually comprise at least one multiple cloning site. In certain embodiments there will be a cloning site or multiple cloning site situated between the promoter and humanised apoaequorin gene. Such cloning sites can be used to create N-terminal fusion proteins by cloning a second nucleic acid sequence into the cloning site so that it is contiguous and in-frame with the humanised apoaequorin gene sequence. In other embodiments there may be a cloning site or multiple cloning site situated immediately downstream of the humanised apoaequorin gene to facilitate the creation of C-terminal fusions in a similar fashion to that for N-terminal fusions described above.

Expression and purification of the polypeptides of the invention can be easily performed using methods well known in the art (for example as described in Sambrook et al. “Molecular Cloning—A Laboratory Manual, second edition 1989”). The construction and use of expression vectors and plasmids is well known to those of skill in the art. Virtually any mammalian cell expression vector can be used to express the codon-optimised apoaequorin nucleic acid sequences of the invention.

According to a further aspect of the invention there is provided a humanised nucleic acid sequence encoding apoaequorin protein, wherein said nucleic acid is positioned under the transcriptional control of a promoter operative in a mammalian cell.

According to a further aspect of the invention there is provided an expression vector comprising a humanised apoaequorin gene and regulatory control sequences capable of directing expression of the humanised apoaequorin gene in a mammalian cell.

The vectors containing the codon-optimised DNA coding for the apoaequorin can be introduced (i.e transformed or transfected) into mammalian, such as CHO; bacterial, such as

E. coli; yeast, such as Saccharomyces cerevisiae or Pichia pastoris; or any other suitable host to facilitate their manipulation (i.e. for mutagenesis, cloning or expression). Performance of the invention is neither dependent on nor limited to any particular strain of host cell or vector; those suitable for use in the invention will be apparent to, and a matter of choice for, the person skilled in the art.

Host cells transformed or transfected with a vector containing an codon-optimised apoaequorin nucleotide sequence may be cultured under conditions suitable for the expression and recovery of the encoded proteins from the cell culture. Such expressed proteins/polypeptides may be secreted into the culture medium or they may be contained intracellularly depending on the sequences used, i.e. whether or not suitable secretion signal sequences were present. Both transient and stably transfected cells/cell lines are contemplated.

Examples of suitable host cells for use in recombinant expression of apoaequorin include, CHO, COS, HeLa, BHK, Vero, MDCK, HepG2, 293, K562, and the like.

Suitable expression systems can also be employed to create transgenic animals capable of expressing aequorin (see for example, U.S. Pat. No. 5,714,666).

According to a further aspect of the invention there is provided a transgenic, non-human animal whose cells comprise a humanised apoaequorin gene and regulatory control sequences capable of directing expression of the humanised apoaequorin gene in said cells.

In a preferred embodiment the transgenic animal is a mouse.

According to a further aspect of the invention there is provided a host cell adapted to express an apoaequorin polypeptide from the codon-optimised nucleic acid sequence of the invention. Preferred host cells are mammalian such as CHO-K1 or Phoenix cells. Human cells are most preferred for expression purposes.

In certain embodiments, the recombinant host cell(s) will express the codon-optimised apoaequorin gene to produce the encoded protein in amounts sufficient to allow luminescent detection of the expressed aequorin.

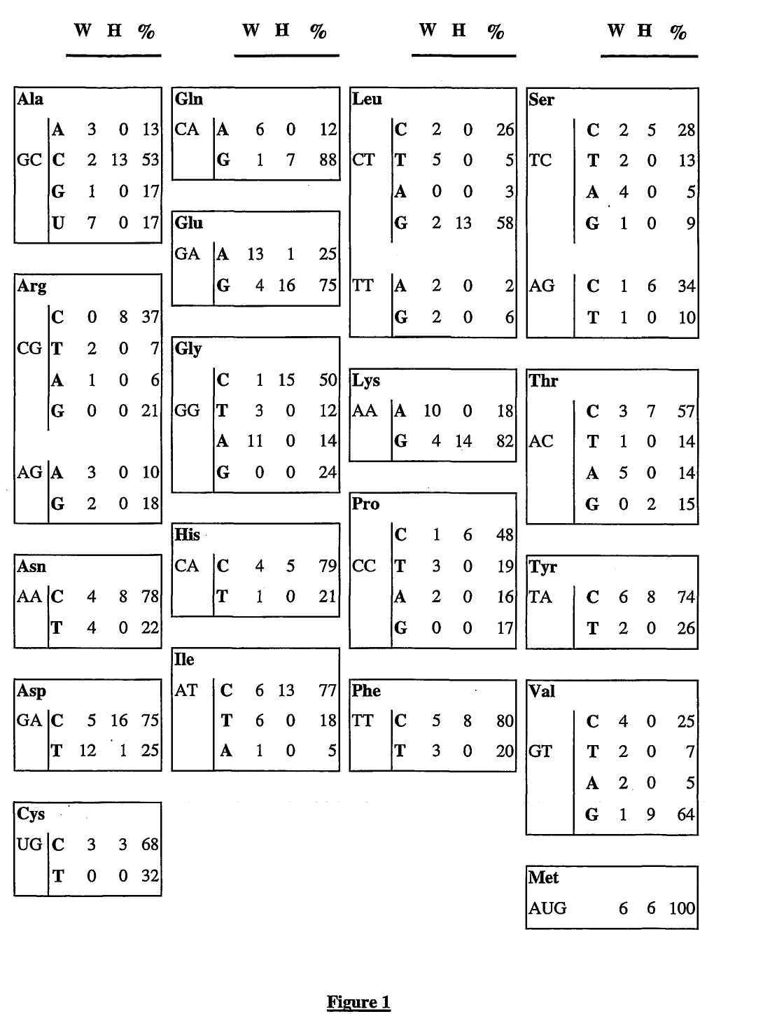

Examination of the codon usage table (FIG. 1) constructed from the native (wt) coding sequence shows that the native jellyfish apoaequorin codons favour either A or U in the third position. Mammalian codon preference generally favours either C or G, and most preferably C, in the third position, regardless of the identity of the two residues in positions 1 and 2. Comparison of 100 highly expressed human genes reveals this tendency, which is graphically presented in FIG. 1 of Haas et al., (Current Biology. 6(3):3135-324, 1996). For example, 53% of Ala (GCX) residues in highly expressed genes are coded for by GCC, 17% by GCT, 13% by GCA and 17% by GCG. Similarly, Ser residues have the following statistics: TCC (28%), TCT (13%), TCA (5%), TCG (9%), AGC (34%) and AGT (10%). As will be apparent from the table of codon-usage between jellyfish and humans, in the amino acids for which there is only a choice of two codons, the wild-type jellyfish apoaequorin gene usually employs the least favoured codon compared to that favoured by human genes.

In constructing the codon optimised apoaequorin DNA, each codon was changed to the mammal equivalent by replacing the third position with either C or G. When this was not possible, due to the introduction of problematic restriction enzyme sites, the next frequently used nucleotide in highly expressed human genes was used.

The codon-optimised apoaequorin genes encompassed by the present invention preferably include those which comprise an increased number of GCC Alanine-encoding codons in comparison to those codons encoding the same amino acid present in the wild-type jellyfish sequence depicted in SEQ ID No. 1.

It will be understood that the term “increased number” when used in this context does not mean that there are for example, more Alanine amino acids present in the codon-optimised form but that, for example, the relative number of GCC Alanine-encoding codons is greater in the codon-optimised apoaequorin form. Of course, this does not exclude there from being numerically more of a particular amino acid within a particular apoaequorin protein compared to the wild-type protein. For example, a variant apoaequorin protein may possesses numerically more of a particular amino acids relative to the wild-type polypeptide sequence depicted in SEQ ID No. 1.

In light of the definition above, the codon-optimised apoaequorin genes encompassed by the present invention preferably include those which comprise an increased number of CGC Arginine-encoding codons; and/or an increased number of AAC Asparagine-encoding codons; and/or an increased number of GAC Aspartate-encoding codons; and/or an increased number of CAG Glutamine-encoding codons; an increased number of GAG Glutamate-encoding codons; and/or an increased number of GGC Glycine-encoding codons; and/or an increased number of CAC Histidine-encoding codons; and/or an increased number of ATC Isoleucine-encoding codons; and/or an increased number of CTG Leucine-encoding codons; and/or an increased number of AAG Lysine-encoding codons; and/or an increased number of CCC Proline-encoding codons; and/or an increased number of TTC Phenylalanine-encoding codons; and/or an increased number of TCC or AGC Serine-encoding codons; and/or an increased number of ACC or ACG Threonine-encoding codons; and/or an increased number of TAC Tyrosine-encoding codons; and/or an increased number of GTG Valine-encoding codons, in comparison to the wild-type jellyfish apoaequorin gene sequence of SEQ ID No.2.

A preferred codon-optimised apoaequorin gene has the sequence depicted in SEQ ID No.3. The codon-optimised genes of the present invention may be fused to other protein/polypeptide-encoding nucleic acid sequences. This will generally result in the expression of a fusion protein in a host cell containing this sequence and regulatory sequences capable of expressing the protein. Both N- and C-terminal fusion proteins are contemplated. The polypeptide fused to the apoaequorin protein may be a secretory or other regulatory sequence, a tag sequence (e.g. 6-his tag), a targeting sequence, and the like.

Alternatively, it is the apoaequorin polypeptide which is serving as a reporter or other marker. An example of a suitable polypeptide for fusion onto the apoaequorin protein is the HA1 haemagglutinin epitope. This can serveas a recognition sequence to verify expression and concentration of the apoaequorin.

According to a further aspect of the invention there is a method for producing apoaequorin protein comprising the steps of:

(i) preparing a recombinant expression vector in which a humanised apoaequorin gene is positioned under the regulatory control of a promoter operative in a mammalian host cell;

(ii) introducing said recombinant expression vector into a suitable mammalian host cell;

(iii) culturing the host cell under conditions suitable for allowing expression of the encoded apoaequorin protein; and optionally,

(iv) purifying said expressed apoaequorin protein from a significant amount of other cellular proteins.

In a preferred embodiment, after step (iv), the expressed apoaequorin protein is at least 70%, more preferably at least 85% and still more preferably at least 95% pure.

According to a further aspect of the invention there is provided a method of increasing the magnitude of aequorin luminescence comprising, introducing into a host cell nucleic acid comprising a humanised nucleic acid sequence coding for apoaequorin polypeptide operably linked to regulatory sequences capable of effecting expression of the humanised nucleic acid to produce said apoaequorin polypeptide. In a preferred embodiment the nucleic acid comprising a humanised nucleic acid sequence coding for apoaequorin polypeptide operably linked to regulatory sequences is introduced into said host cells by transfection, transformation or electroporation. In a further embodiment, the host cells are grown in condition suitable for expression of said introduced apoaequorin nucleic acid. It will be appreciated that by “increasing the magnitude of aequorin luminescence”, the increase in magnitude is relative to the same expression system but wherein the native (non-humanised) jellyfish apoaequorin gene is used.

According to a further aspect of the invention there is provided the use of a codon-optimised apoaequorin nucleic acid sequence for enhancing the magnitude of aequorin luminescence in a host cell.

According to a further aspect of the invention there is provided a method for measuring the ability of a compound to block/inhibit/antagonise receptors, such as G-protein coupled receptors (GPCR) or ion channels, which mediate changes in intracellular calcium flux when activated. For example, a GPCR can be expressed in cell line, such as HEK293 or CHO cells, engineered to express codon optimised apoaequorin. The apoaequorin is converted to aequorin by incubation of the cells with the luciferin coelenterazine prior to the assay. A test compound is added to the cells, followed by a ligand. Luminescence produced by increased calcium flux due to receptor activation is then measured by a standard luminometer. The luminescent output of the cells treated with compound is then compared to those treated with ligand alone to calculate the degree of inhibition caused by the test compound. Alternatively, receptor agonists can be found by treating the aequorin cells with test compounds and directly measuring luminescent output.

Thus, in accordance with this aspect of the invention, mammalian cells expressing a receptor involved in the modulation of intracellular calcium and engineered to express aequorin from a humanised gene are incubated with a compound of interest. Coelenterazine cofactor is added and photon emission is measured whereby the emission of photons is indicative of the amount of intracellular calcium release. According to a further aspect, the results of the test, i.e the ability of the test compound(s) to inhibit or modulate the receptor, is recorded, for example on paper, and the like, or electronic means.

The invention will be further described by reference to the following non-limiting Examples and figures, in which:

FIG. 1—is a table listing the codon-usage of the wild-type jellyfish apoaequorin gene (W), the humanised apoaequorin gene (H) generated in Example 1, and the percentage (%) of codon usage in 100 highly expressed human genes (adapted from Haas et al., Current Biology. Vol. 6(3):315-324, 1996).

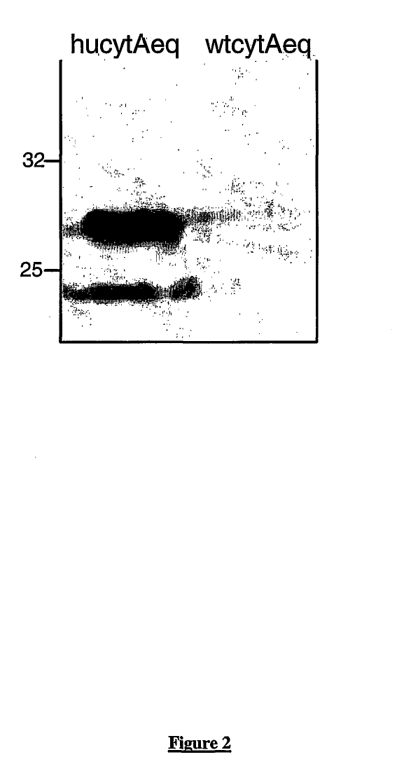

FIG. 2—is a western blot showing the elevated level of expression of humanised aequorin (@8-fold) compared to non-humanised aequorin.

### MATERIALS

The plasmid comprising cytoplasmic-targeting wild-type apoaequorin gene, cytAEQ/pcDNA1 was purchase from Molecular Probes (Europe). This vector contains sequences encoding the HA1 haemagglutinin epitope (YPYDVPDYA Seq ID No: 5) fused to the apoaequorin structural gene in the expression vector pcDNA1. Synthetic coelenterazine derivative designated hcp was purchased from Molecular Probes (Europe). This hcp coelenterazine derivative, when reconstituted with apoaequorin, shows very favourable characteristics, including a fast response to binding Ca²⁺ and the highest luminescence quantum yield as compared with the other four commercially available derivatives.

### METHODS

Plasmid Construct

The 660 bp EcoR1 apoaequorin fragment from cytaeqpcDNA1 was cloned into pcDNA3 (Invitrogen) to form plasmid cytaeqpcDNA3#2. This was used as a control in the experiments that followed. The nucleotide sequence of wild-type apoaequorin is depicted in SEQ ID NO:2.

Codon optimisation of the entire apoaequorin coding sequence was essentially performed as described in Haas et al., (Current Biology. 6(3):315-324, 1996). Briefly, the codon optimised apoaeqorin sequence was assembled from six fragments of approximately 120 bp each, generated from long synthetic oligonucleotides containing portions of the apoaequorin coding sequence flanked by BsaI sites at one end and BbsI sites at the other end, in the configuration BsaI-apoaequorin-BbsI. The synthetic DNA segments containing the codon optimised apoaequorin sequence were amplified by PCR, cloned in pCRscript(cam) (Stratagene) and sequenced to confirm correct DNA sequence. The intact apoaequorin coding sequence was then assembled by sequential ligation of the BsaI-BbsI fragments. The codon-optimised version was designated cythuaeqHA1#20. The nucleotide sequence of the codon-optimised sequence is depicted in SEQ ID NO: 3. A full length HA1 epitope (SEQ ID No. 4) was then cloned onto the 5′ end of the codon-optimised apoaequorin gene to form. cythuaeqHA1#20 (9aa).

Plasmids for Transient Experiments.

The cythuaeqHA1#20 (9aa) PCR fragment was blunt end cloned into Srf restriction site of PCR Script(cam) plasmid (Stratagene), transformed into Life technologies DH5α Ultra Competent cells and cultured overnight at 37° C. on 50 μg/ml chloramphenicol plates +100 μl 10 mM IPTG and 100 μl 2% X-gal. Chloramphenicol resistant colonies were screened for inserts by EcoR1 restriction endonuclease digestion. The positive colonies were sequenced to confirm correct sequence and orientation. Typically, 2 μg of Miniprep plasmid DNA was sequenced using approximately 6.4 pmoles T7 or T3 primer. The cythuaeqHA1 inserts were rescued from PCRScript(cam) by restriction endonuclease digestion with EcoR1. Agarose gel electrophoresis was used to separate the apoaequorin inserts from the plasmid. The 660 bp inserts were excised, gel purified and then ligated into EcoR1 linearised and dephosphorylated pcDNA3. The ligation mixture was used to transform DH5α Ultra competent cells. Ampicillin resistant colonies were screened for inserts using EcoR1 restriction endonuclease digestion. Insert orientation was confirmed by restriction analysis using HindIII and BamH1 restriction enzymes. The plasmid comprising the humanised apoaequorin gene/HA1 fusion in the correct orientation was designated hucytaeqHA(9aa)pcDNA3#20.

Cell Culture and Transfection

CHOK1 and Phoenix (HEK293 derived) cells were grown in Ham's F12 and DMEM respectively. The medium was supplemented with10% foetal calf serum, 2 mM glutamine, penicillin and streptomycin 100 μg/ml.

Cells were seeded in Costar 6 well plates at a density of 5×10⁵ 18-24 hours prior to transfection. On the day of transfection the cells were 85-90% confluent. Plasmids, cytaeqHA1(9aa)pcDNA3#2 and hucytaeqHA(9aa)pcDNA3#20, were transfected into the cells using Lipofectamine Plus reagents. A co-transfection with EGFP (Clontech) allowed transfection variation between wells to be ascertained. A modification to the standard protocol from Life Technologies was used. In summary, 2 μg of DNA (1 μg of each for co-transfections) diluted in 400 μl of HamsF12+12 μl lipofectamnine plus reagent was incubated for 15 minutes at room temperature. 8 μl of Lipofectamine reagent +400 μl of HamsF12 medium was also incubated for 15 minutes at room temperature. The two mixtures were combined and incubated for a further 15 minutes before being aspirated onto the cells containing 1.6 mls of Ham's F12 medium. The plates were incubated for 3 hours at 37° C. 5% CO₂. The transfection reagents were replaced with Ham's F12 complete medium and the cells incubated for 24 hours. At 24 hours the cells were transferred to 96 well plates. 48 hours post transfection the 96 wells were rinsed in PBS and replaced with 50 μl of DMEM (without phenol red) containing 1% heat inactivated FCS and 2.5 μm hcp coelenterazine. The cells were incubated at 37° C. 5% CO₂ for 3 hours. The wells were aspirated of the coelenterazine mix and replaced with 175 μl of PBS containing either 1 μM or 2 μM CaCl₂. The plates were transferred to Tropix TR717 luminometer for stimulation. 2 μM of ionomycin (25 μl), a calcium ionophore, was injected into the wells and the reaction recorded over 60 seconds. The results are recorded in Table. 1:

TABLE 1

Peak Relative Light Units (RLU) following
Ionomycin Stimulation (2 mM)

| Aequorin | 1 μM CaCl₂ | 2 μM CaCl₂ |
|---|---|---|
| Codon optimised | 18008 (19) | 15561 (21) |
| Wild type | 944 | 734 |

illustrates calcium flux detected within HEK293 cells transiently transfected with either codon optimised or wild type apoaequorin expression plasmids, stimulated 48 hours post transfection with Ionomycin, demonstrating a 19-21-fold increase in luminescent output (RLU) with the codon optimised aequorin. Numbers in parenthesis represent the fold increase in RLU relative to wild type aequorin.

Immunoblotting

Expression of recombinant protein was verified by immuoblotting using Anti-HA-Peroxidase, High Affinity (3F10) monoclonal antibody (Roche). This antibody recognizes the HA peptide sequence YPYDVPDYA derived from the human hemagglutinin protein (Kolodziej &Young, Methods Enzymol. 194:508-511, 1191). HEK 293 cells were transiently transfected with the codon optimised and the wild type aequorin constructs. Cell lysates were prepared 48 hours post transfection, proteins (130 μg) separated by SDS-PAGE electrophoresis, transferred to nylon membrane and stained with a 1:500 dilution of 3F10 antibody. Protein expression was quantitated using pixel quantification in ImageQuant software (Molecular Dynamics) and indicated a 8-fold increase in expression of cythuAEQ over cytAEQ.

1. A codon-optimised nucleic acid sequence coding for apoaequorin polypeptide.

2. A codon-optimised nucleic acid as claimed in claim 1, wherein said codon-optimised nucleic acid encodes an apoaequorin polypeptide that has the amino acid sequence depicted in SEQ ID No. 1, a variant thereof or truncated versions of either.

3. A codon optimised nucleic acid as claimed in claim 1, wherein said nucleic acid comprises an increased number of GCC Alanine-encoding codons; and/or an increased number of CGC Arginine-encoding codons; and/or an increased number of AAC Asparagine-encoding codons; and/or an increased number of GAC Aspartate-encoding codons; and/or an increased number of CAG Glutamine-encoding codons; an increased number of GAG Glutamate-encoding codons; and/or an increased number of GGC Glycine-encoding codons; and/or an increased number of CAC Histidine-encoding codons; and/or an increased number of ATC Isoleucine-encoding codons; and/or an increased number of CTG Leucine-encoding codons; and/or an increased number of AAG Lysine-encoding codons; and/or an increased number of CCC Proline-encoding codons; and/or an increased number of TTC Phenylalanine-encoding codons; and/or an increased number of TCC or AGC Serine-encoding codons; and/or an increased number of ACC or ACG Threonine-encoding codons; and/or an increased number of TAC Tyrosine-encoding codons; and/or an increased number of GTG Valine-encoding codons, in comparison to the wild-type jellyfish apoaequorin gene sequence of SEQ ID No. 2.

4. A codon optimised nucleic acid as claimed in claim 1, wherein said nucleic acid is positioned under the transcriptional control of a promoter operative in a mammalian cell.

5. An expression vector comprising a humanised apoaequorin gene and regulatory control sequences capable of directing expression of the humanised apoaequorin gene in a mammalian cell.

6. A recombinant host cell comprising a humanised apoaequorin gene.

7. A recombinant host cell as claimed in claim 6, which is a mammalian, preferably human cell.

8. A recombinant host cell as claimed in claim 6, wherein said cell is located in a non-human mammal.

9. A method for producing apoaequorin protein comprising the steps of:
(i) preparing a recombinant expression vector in which a humanised apoaequorin gene is positioned under the regulatory control of a promoter operative in a mammalian host cell;
(ii) introducing said recombinant expression vector into a suitable mammalian host cell;
(iii) culturing the host cell under conditions suitable for allowing expression of the encoded apoaequorin protein; and optionally, purifying said expressed apoaequorin protein from a significant amount of other cellular proteins.

10. A method of increasing the magnitude of aequorin luminescence comprising, introducing into a host cell nucleic acid comprising a codon-optimised nucleic acid sequence coding for apoaequorin polypeptide operably linked to regulatory sequences capable of effecting expression of the codon-optimised nucleic acid to produce said apoaequorin polypeptide.

11. The use of a codon-optimised apoaequorin nucleic acid sequence for enhancing the magnitude of aequorin luminescence in a host cell.

12. A method for measuring the ability of a compound to inhibit a receptor which mediates changes in intracellular calcium flux when activated, comprising contacting a host cell engineered to express codon optimised apoaequorin with the luciferin coelenterazine and measuring the amount of luminescence produced with or without addition of a test compound.

13. The method as claimed in claim 12, further comprising recording the receptor inhibiting ability of the test compound.


SEQUENCE LISTING (5 sequences)

SEQ ID NO: 1 - protein, 196 aa, Aequorea victoria
MTSEQYSVKLTPDFDNPKWIGRHKHMFNFLDVNHNGRISLDEMVYKASDIVINNLGATPEQAKRHKDAVEAFFGGAGMKYGVETEWPEYIEGWKRLASEELKRYSKNQITLIRLWGDALFDIIDKDQNGAISLDEWKAYTKSDGIIQSSEDCEETFRVCDIDESGQLDVDEMTRQHLGFWYTMDPACEKLYGGAVP

SEQ ID NO: 2 - DNA, 570 bp, Aequorea victoria
atgaagcttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacaacatttaggattttggtacaccatggatcctgcttgcgaaaagctctacggtggagctgtcccctaa

SEQ ID NO: 3 - DNA, 570 bp, Artificial Sequence
  humanised apoaequorin
atgaagctgacccccgacttcgacaaccccaagtggatcggccgccacaagcacatgttcaacttcctggacgtgaaccacaacggccgcatcagcctggacgagatggtgtacaaggccagcgacatcgtgatcaacaacctgggcgccacccccgagcaggccaagcgccacaaggacgccgtggaggccttcttcggcggcgccggcatgaagtacggcgtggagacggagtggcccgagtacatcgagggctggaagcgcctggcctccgaggaactgaagcgctacagcaagaaccagatcaccctgatccgcctgtggggcgacgccctgttcgacatcatcgacaaggaccagaacggcgccatctccctggatgagtggaaggcctacaccaagtccgtcggcatcatccagtcctccgaggactgcgaggagacgttccgcgtgtgcgacatcgacgagagcggccagctggacgtggacgagatgacccgccagcacctgggcttctggtacaccatggaccccgcctgcgagaagctgtacggcggcgccgtgccctaa

SEQ ID NO: 4 - DNA, 27 bp, Artificial Sequence
  HA1 epitope
tacccctacgacgtgcccgactacgcc

SEQ ID NO: 5 - protein, 9 aa, Artificial Sequence
  HA1 epitope
YPYDVPDYA